(19) United States
(12) Patent Application Publication
(10) Pub. No.: US 2012/0165704 A1
(43) Pub. Date: Jun. 28, 2012

(54) APPARATUS FOR CONTROLLING REHABILITATION ROBOT

(75) Inventors: Sang Seung KANG, Daejeon (KR); Eun Hye JANG, Daejeon (KR); Jae Yeon LEE, Daejeon (KR); Suyoung CHI, Daejeon (KR); Young-Jo CHO, Daejeon (KR); Jae Hong KIM, Daejeon (KR); Joo Chan SOHN, Daejeon (KR)

(73) Assignee: Electronics and Telecommunications Research Institute, Daejeon (KR)

(21) Appl. No.: 13/334,564

(22) Filed: Dec. 22, 2011

(30) Foreign Application Priority Data
Dec. 23, 2010 (KR) 10-2010-0133147

(51) Int. Cl. A61B 5/103; A61H 1/02

(52) U.S. Cl. 600/595; 601/23; 601/35

(57) ABSTRACT

An apparatus for controlling a rehabilitation robot providing walking assistance to a user is disclosed. The apparatus includes a command perceiving unit for receiving a explicit walking command mode of a user based on a conscious adjustment signal, a motion sensing unit for sensing action of the user, a biometric sensing unit for sensing a biometric state of the user, and a host control module for analyzing walking steps and perceiving intent to walk by using the walking command perceived by the command perceiving unit and the action sensed by the motion sensing unit, thereby controlling the rehabilitation robot. The apparatus for controlling a rehabilitation robot further includes a wearing-type suit having the apparatus disposed thereon.

### CROSS-REFERENCE TO RELATED APPLICATION(S)

The present invention claims priority of Korean Patent Application No. 10-2010-0133147, filed on Dec. 23, 2010, which is incorporated herein by reference.

### FIELD OF THE INVENTION

The present invention relates generally to an apparatus for controlling a rehabilitation robot and, more particularly, to an apparatus for controlling a rehabilitation robot, which allows a user to easily control the rehabilitation robot in accordance with user's intent to walk by sensing the intent of the user to move.

### BACKGROUND OF THE INVENTION

As is well known to those skilled in the art, a variety of researches have been carried out into rehabilitation robots that reinforce the muscular strengths of the old and the weak or patients or that help them walk. Such rehabilitation robots are also called walk assist robots and may assist users in walking so that paraplegic patients can walk by themselves.

In such rehabilitation robots, it is important to implement a robot control mechanism for performing a walk motion suitable for the intent of the user. In relation to that, research into various technologies for transferring a user's intent to walk to a robot control apparatus has been conducted.

The basic technology for the rehabilitation robots is configured such that a user personally inputs his or her intent to walk using input buttons, but is problematic in that a plurality of input buttons are required to perform various motions and the user having to perform an input action for each motion is inconvenient.

Another technology that has been proposed is a technology for sensing a user's intent to walk using an indirect path rather than using the user's direct input. One example of this technology is a technique for analyzing the information of an encoder for driving a rehabilitation robot and controlling the subsequent motion of the rehabilitation robot on the basis of the analyzed encoder information. This technology may be effective for the repetitive pattern of walking, but is problematic in that it is difficult to apply this technology to other patterns such as stopping or resuming walking.

A further another technology that has been proposed is a technology for analyzing walking patterns using, e.g., a pressure sensor attached to the sole of the foot and controlling a rehabilitation robot based on the analyzed walking patterns. This technology controls the rehabilitation robot by analyzing the degree or distribution of a pressure on the front or rear part of the sole of the foot, but it is problematic in that it is impossible to detect such information from a paraplegic patient.

Yet another technology that has been proposed is a technology for controlling a rehabilitation robot using electromyogram (EMG) signals generated by the motion of a body. Since biometric signals such as EMG signals have a regular pattern, it may be possible to catch the user's intent, but it is difficult to sense the signals and there is a limitation in detecting precise signals due to dynamic noise attributable to a motion when a motion is small.

### SUMMARY OF THE INVENTION

In view of the above, the present invention provides an apparatus for controlling a rehabilitation robot, which controls a rehabilitation robot providing walking assistance in compliance with walk commands directly input by a user, and also controls the rehabilitation robot by sensing the user's unconscious intent to walk.

In accordance with a first aspect of the present invention, there is provided an apparatus for controlling a rehabilitation robot providing walking assistance to a user, comprising:

a command perceiving unit for receiving a explicit walking command mode of a user based on a conscious adjustment signal;

a motion sensing unit for sensing action of the user;

a biometric sensing unit for sensing a biometric state of the user; and

a host control module for analyzing walking steps and perceiving intent to walk by using the walking command perceived by the command perceiving unit and the action sensed by the motion sensing unit, thus controlling the rehabilitation robot.

Preferably, the apparatus for controlling a rehabilitation robot further includes a wearing-type suit having the apparatus disposed thereon.

### BRIEF DESCRIPTION OF THE DRAWINGS

The above and other objects and features of the present invention will become apparent from the following description of preferred embodiments given in conjunction with the accompanying drawings, in which:

FIG. 1 is a block diagram of an apparatus for controlling a rehabilitation robot in accordance with an embodiment of the present invention;

FIG. 2 is a block diagram of an apparatus for controlling a rehabilitation robot, which is mounted on a wearing-type suit, in accordance with a first embodiment of the present invention;

FIG. 3 is a block diagram showing of an apparatus for controlling a rehabilitation robot, which is mounted on a wearing-type suit, in accordance with a second embodiment of the present invention; and

FIG. 4 is a flowchart showing a method for controlling a rehabilitation robot in accordance with an embodiment of the present invention.

### DETAILED DESCRIPTION OF THE PREFERRED EMBODIMENTS

Hereinafter, embodiments of the present invention will be described in detail with reference to the accompanying drawings so that they can be readily implemented by those skilled in the art.

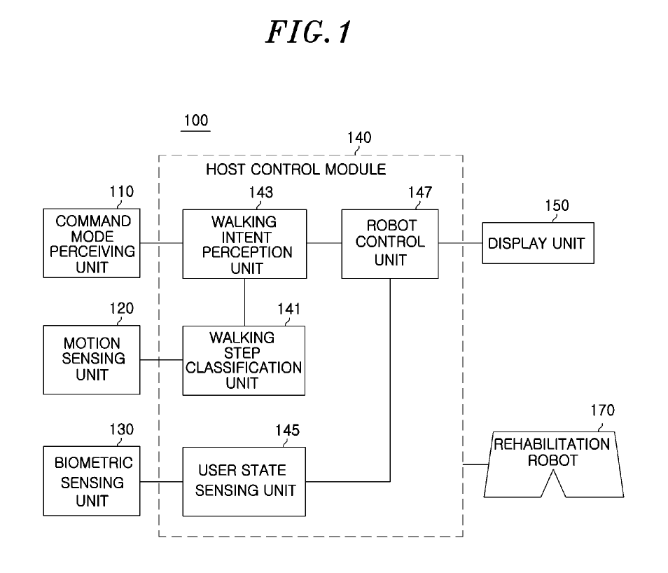

FIG. 1 is a block diagram of an apparatus for controlling a rehabilitation robot in accordance with an embodiment of the present invention.

As shown in FIG. 1, an apparatus 100 for controlling a rehabilitation robot includes a command mode perceiving unit 110, a motion sensing unit 120, a biometric signal sensing unit 130, a host control module 140, a display unit 150 and a rehabilitation robot 170.

The command mode perceiving unit 110 perceives a user's conscious adjustment signal including an explicit walking command provided via an input interface.

The motion sensing unit 120 senses the force or pressure applied to the palm of the hand and the sole of the foot caused by the motion or action of the user using a force sensor or a pressure sensor. Further, the motion sensing unit 120 senses tilt, posture or direction of the user by using an inertial sensor.

The biometric sensing unit 130 monitors physical state of the user to recognize abnormal condition using a biometric sensor.

The host control module 140 analyzes walking steps using both the explicit walking command and the motion signal sensed from the user's motion, perceives the intent of the user to walk, and then controls the rehabilitation robot. More specifically, the host control module 140 perceives a conscious adjustment signal such as the explicit walking command provided by the command perceiving unit 110 and also senses unconscious condition involved in walking by merging a plurality of motion signals sensed by the motion sensing unit 120, thus controlling preceding information when giving an unconscious walking command. Further, the host control module 140 perceives the intent to walk and the state of walking of the user from the motion signals and biometric signals which are caused by the user's motions made once walking has commenced, and perceives the current walking step of the user, thus controlling subsequent information that is to be utilized for predicting subsequent walking steps. The host control module 140 includes a walking step classification unit 141, a walking intent perception unit 143, a user state sensing unit 145, and a robot control unit 147.

The walking step classification unit 141 combines the motion signals with one another, extracts effective feature data from the combined signal, and then classifies the walking steps of the user.

The walking intent perception unit 143 perceives the intent of the user to walk on the basis of the walking steps from the walking step classification unit 141 and the explicit walking command input by the user.

The user state sensing unit 145 analyzes biometric signals sensed by the biometric sensing unit 130, extracts effective feature data, and determines the physical state of the user, to thereby recognize the physical state of the user.

The robot control unit 147 controls the rehabilitation robot 170 so that walking suitable for the perceived user's intent to walk can be performed when the state of the user is stable.

The display unit 150 displays various types of information. For example, the display unit 150 displays information related to the commands input through the input interface, the current walking step, the biometric signals and processed information thereof, and the status information for system management.

The walking step classification unit 141 and the user state sensing unit 145 may arrange motion signals and biometric signals, which are suitable for walking patterns of a user, for example, a paralegic patient, into a database (DB), in order to perform a learning and then determine a walking step and state of the paralegic patient on the basis of previously learned data.

Further, if it is determined that the physical state of the user recognized by the user state sensing unit 145 is beyond a predetermined stable condition, a warning message, for example, a warning sound or a danger notifying sound may be output via the display unit 150 or the warning message may be provided in the form of a vibration via the display unit 150. If a state of an abnormal condition is perceived, the robot control unit 147 stops the walking step of the rehabilitation robot 170, and performs a control so that the rehabilitation robot 170 is driven within a predefined stable condition.

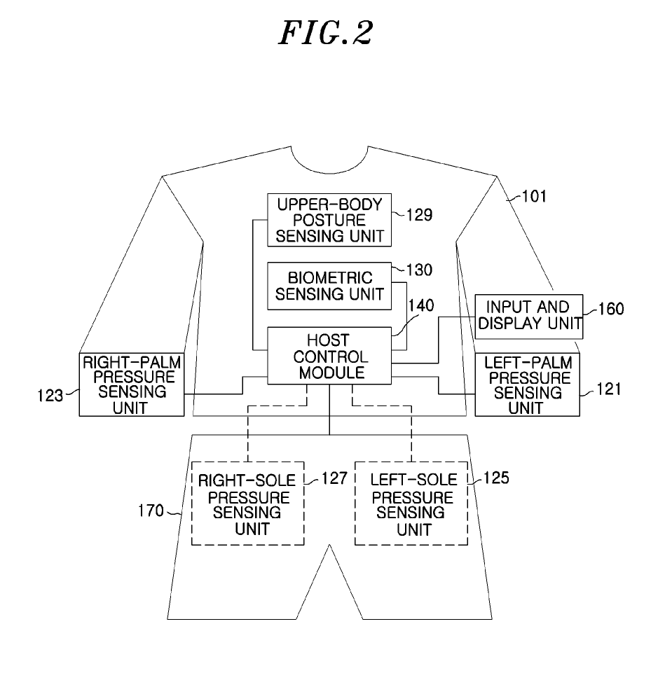

FIG. 2 is a block diagram of an apparatus for controlling a rehabilitation robot, which is mounted on a wearing-type suit, in accordance with a first embodiment of the present invention.

Referring to FIG. 2, the rehabilitation robot controlling apparatus includes a rehabilitation robot 170 and a wearing-type suit 101.

The rehabilitation robot 170 may be mounted on the legs of the user, e.g., a paraplegic patient to assist the walking steps of the user. The wearing-type suit 101 may be implemented in the form of an upper garment worn on the upper body of the user. Moreover, wearing-type suit 101 may be implemented using various types of upper garments, such as a long-sleeved jacket, a short-sleeved jacket, a vest, or top underwear.

The rehabilitation robot controlling apparatus also includes an input and display unit 160, a left-palm pressure sensing unit 121, a right-palm pressure sensing unit 123, a left-sole pressure sensing unit 125, a right-sole pressure sensing unit 127, and an upper-body posture sensing unit 129, a biometric sensing unit 130, and a host control module 140.

The command perceiving unit 110 and the display unit 150 of FIG. 1 is embodied as the input and display unit 160, that are integrated as one unit. Further, the motion sensing unit 120 of FIG. 1 is separated into and embodied as components, such as the left-palm pressure sensing unit 121, the right-palm pressure sensing unit 123, the left-sole pressure sensing unit 125, the right-sole pressure sensing unit 127, and the upper-body posture sensing unit 129. Alternatively, the left-palm pressure sensing unit 121 or the right-palm pressure sensing unit 123 may be implemented to be integrated with the input and display unit 160.

In this embodiment, among the components of the rehabilitation robot controlling apparatus, the host control module 140 may be disposed in the wearing-type suit 101. Some or all of the input and display unit 160, the left-palm pressure sensing unit 121, the right-palm pressure sensing unit 123, the upper body posture sensing unit 129, and the biometric sensing unit 130 may also be disposed in the wearing-type suit 101.

The left-sole pressure sensing unit 125 and the right-sole pressure sensing unit 127 may be separately installed on the legs on which the rehabilitation robot 170 is mounted. In this case, they may be connected to the host control module 140 in a wired or wireless communication manner. In addition, the wearing-type suit 101 may be configured to include a power control unit and a battery for supplying power to the components of the apparatus 200 inclusive of the rehabilitation robot 170.

The host control module 140 controls the rehabilitation robot 170 by analyzing a walking step and perceiving the user's intent to walk using both a walking command input by the user and motion signals sensed from the action of the user. The host control module 140 perceives the user's intent to walk and monitors the physical state of the user based on biometric signals and action of the user via interaction with the individual components. In this case, the components may be operated to be separated into individual modules, and may perform a communication function in a wired or wireless manner so as to communicate with the individual components.

The input and display unit 160 provides an input interface for allowing the user to explicitly input a variety of commands so as to select a walking type, and represents various types of information including the state of the user. The explicit commands may be presented to select walking, standing, sitting, going up stairs, going down stairs, stopping, or the like. The input interfaces may include a type that uses a key or a button, a type that uses a touch interface based on a touch panel, and a combination of the two types. Further, the input and display unit 160 may display the commands modes input through the input interface, the information related to the current walking step, the biometric signals and processed information thereof, and the status information for system management. Furthermore, the input and display unit 160 may include a sound output unit, a vibration output unit, etc. for representing information fed back from interaction with the host control module 140.

The left-palm pressure sensing unit 121 and the right-palm pressure sensing unit 123 may include one or more force sensors or pressure sensors located on the palms of the respective hands using gloves or the like, or are mounted on portions of clutches to which the palms of the hands touch. Here, the clutches are used in such a way that when a paraplegic patient walks, the patient grasps the clutches with the hands to create a walking command. Accordingly, when the paraplegic patient moves while grasping the clutches, the magnitude of force or pressure applied to the palms of the hands can be measured. The magnitude of the force or pressure may be normalized or scaled for the subsequent processing. In a case where the left-palm pressure sensing unit 121 and the right-palm pressure sensing unit 123 are mounted on the palms of the hands via gloves or the like, they may communicate with the host control module 140 in a wired or wireless manner. Further, in a case where the left-palm and the right-palm pressure sensing units 121 and 123 are mounted on the clutches, they may communicate with the host control module 140 in a wireless manner.

The upper-body posture sensing unit 129 is implemented as one or more of inertial sensors such as an acceleration sensor, an angular velocity sensor, or a terrestrial magnetism sensor. The upper-body posture sensing unit 129 senses at least one of the tilt, posture and direction of the upper body in accordance with the motion of the user, to recognize the motion about the upper body of the user.

The left-sole pressure sensing unit 125 and the right-sole pressure sensing unit 127 may include force sensors or pressure sensors respectively disposed on the soles of the left foot and the right foot of the legs of the user on which the rehabilitation robot 170 is mounted, thus sensing the degree of pressure applied to the soles of the feet when the user walks. Even if the left-sole pressure sensing unit 125 and the right-sole pressure sensing unit 127 are not directly installed in the wearing-type suit 101, they may be mounted in the form of the insoles of shoes equipped with force sensors or pressure sensors via the rehabilitation robot 170 mounted on the legs so as to sense the degree of pressure applied to the soles of the left foot and the right foot.

The biometric sensing unit 130 analyzes the physical state of the user and predicts abnormal condition using a biometric sensor such as an electrocardiogram, a pulse wave, a body temperature sensor, etc. More specifically, the biometric sensing unit 130 measures biometric signals including a heartbeat, pulsation and body temperature using the biometric sensor, analyzes the physical state of the user and recognizes the abnormal condition from the biometric signals. The biometric sensing unit 130 may further include a piezoelectric sensor so as to detect excessive pressure that may be put on the user.

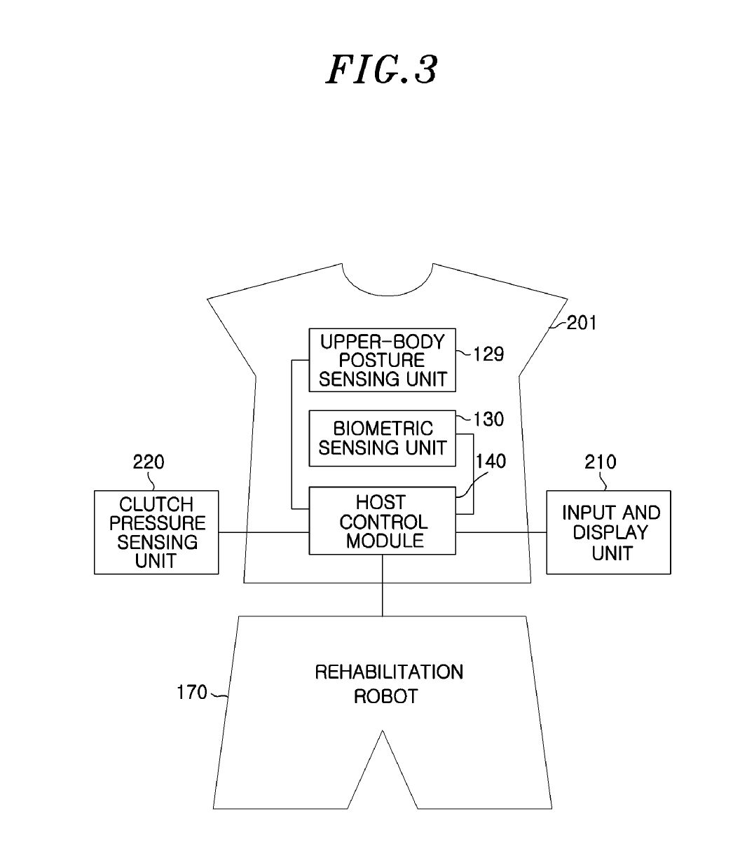

FIG. 3 is a block diagram of an apparatus for controlling a rehabilitation robot, which is mounted on a wearing-type suit, in accordance with a second embodiment of the present invention

Referring to FIG. 3, the rehabilitation robot controlling apparatus includes an upper-body posture sensing unit 129, a biometric signal sensing unit 130 and a host control module 140 that are disposed on a wearing-type suit 201. The rehabilitation robot controlling apparatus also includes an input and display unit 210 and a clutch pressure sensing unit 220 that are not disposed on the wearing-type suit 201 but are implemented as separate devices. Therefore, the input and display unit 210 and the clutch pressure sensing unit 220 are configured to transmit or receive data to or from the host control module 140 using wireless communication.

By comparison of the components of the embodiment of FIG. 3 with those of the embodiment of FIG. 2, the clutch pressure sensing device 220 corresponds to a set of the left-palm pressure sensing unit 121, the right-palm pressure sensing unit 123, the left-sole pressure sensing unit 125, and the right-sole pressure sensing unit 127. Further, the input and display unit 210 corresponds to the input and display unit 160.

The input and display unit 210 is attached to the wrist of a user and facilitates the user to input various commands and check information displayed thereon. The clutch pressure sensing unit 220 measures the degree of force or pressure applied to the palms of the hands grasping clutches to create a walking command when a user, e.g., a paraplegic patient walks.

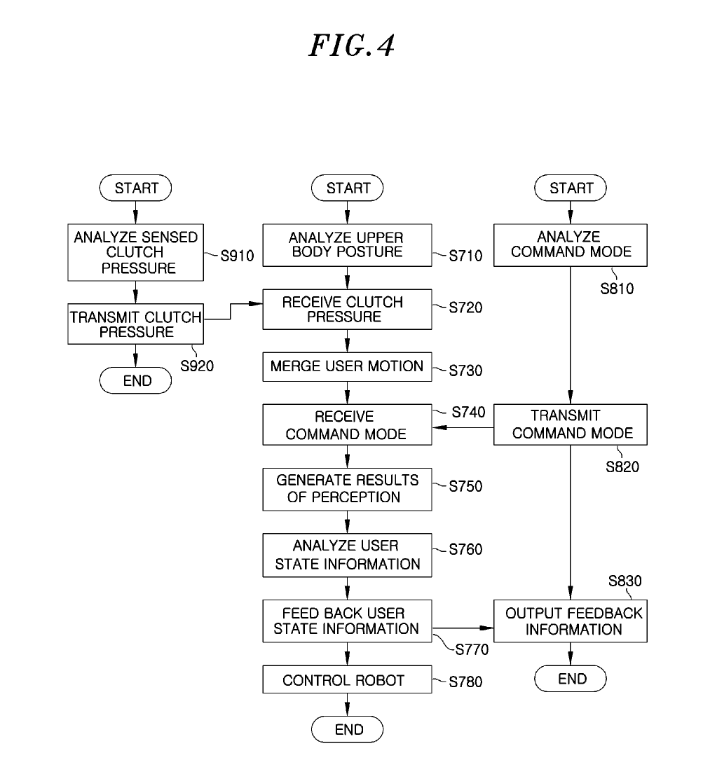

FIG. 4 is a flowchart illustrating a method for controlling a rehabilitation robot in accordance with an embodiment of the present invention with reference to FIG. 1.

First, at step S710, the host control module 140 analyzes the upper body posture provided from the motion sensing unit 120 to create posture data.

Meanwhile, the motion sensing unit 120 senses the clutch pressure on the left palm or on the right palm of the user step S910, and transmits the sensed clutch pressure to the host control module 140 at step S920.

At step S720, the host control module 140 receives the clutch pressure.

At step S730, the host control module 140 merges the clutch pressure and the posture data.

Meanwhile, the command mode perceiving unit 110 analyzes information about the command input by the user at step S810, and transmits the command to the host control module 140 at step S820.

At step S740, the host control module 140 receives the command from the command perceiving unit 110.

Thereafter, at step S750, the host control module 140 classifies the walking steps of the user depending on the command, analyzes the user's intent, and generates the results of perception.

Next, the host control module 140 analyzes the physical state about the user on the basis of biometric signals sensed by the biometric sensing unit 130 at step S760.

The analyzed physical state is provided to the display unit 150 as feedback information at step S770. The display unit 150 displays the feedback information received from the host control module 140 so that the user may be aware of the feedback information at step S830.

Finally, at step S780, the host control module 140 controls the rehabilitation robot 170 if it is determined that the state information fulfills predetermined stable conditions.

In accordance with embodiments of the present invention, a user' intent to move is sensed, and the physical state of the user is monitored and transmitted as feedback information, so that the user of the rehabilitation robot providing walking assistance to a paraplegic patient can easily control the rehabilitation robot in accordance with his or her intent to walk, and can minimize the inconvenience of having to separately mount a plurality of sensor modules, by using a wearing-type suit.

Further, in the case of an apparatus for controlling a rehabilitation robot in which modules are separately implemented, an easy configuration method for increasing the convenience of the user wearing the apparatus is provided, thus enabling various approaches to be implemented.

Consequently, the embodiments of the present invention are advantageous in that it is possible for the walking of a rehabilitation robot to satisfy both the explicit walking command and the implicit walking intent of the user.

While the invention has been shown and described with respect to the preferred embodiments, it will be understood by those skilled in the art that various changes and modifications may be made without departing from the spirit and scope of the invention as defined in the following claims.

What is claimed is:

1. An apparatus for controlling a rehabilitation robot providing walking assistance to a user, comprising:
a command perceiving unit for receiving a explicit walking command mode of a user based on a conscious adjustment signal;
a motion sensing unit for sensing action of the user;
a biometric sensing unit for sensing a biometric state of the user; and
a host control module for analyzing walking steps and perceiving intent to walk by using the walking command perceived by the command perceiving unit and the action sensed by the motion sensing unit, thus controlling the rehabilitation robot.

2. The apparatus of claim 1, wherein the motion sensing unit comprises a palm pressure sensing unit for sensing a pressure applied to a clutch, which is grasped by the hand to input the walking command.

3. The apparatus of claim 1, wherein the motion sensing unit comprises an upper-body posture sensing unit for sensing the tilt and posture of an upper body of the user.

4. The apparatus of claim 1, wherein the motion sensing unit comprises a sole pressure sensing unit for sensing a pressure applied to the sole of the user while walking.

5. The apparatus of claim 1, wherein the host control module comprises:
a walking step classification unit for combining the motion signals sensed by the motion sensing unit, extracting effective feature data from the combined signals, and then classifying walking steps of the user;
a walking intent perception unit for perceiving the intent of the user to walk from the walking steps, and the explicit walking command;
a user state sensing unit for analyzing the biometric state sensed by the biometric sensing unit, extracting effective feature information, and then recognizing the physical state of the user; and
a robot control unit for controlling the rehabilitation robot so that it can perform walking suitable for the perceived intent of the user to walk when the state of the user is stable.

6. The apparatus of claim 5, wherein the walking step classification unit arranges information about the action suitable for walking patterns of the user into a database in order to perform learning, and then determine the walking step based on previously learned data.

7. The apparatus of claim 6, wherein the user state sensing unit arranges information about the biometric state suitable for walking patterns of the user into a database in order to perform a learning, and then determines the state information based on previously learned data.

8. The apparatus of claim 6, further comprising a display unit for displaying feedback information including the biometric state under a control of the host control module.

9. The apparatus of claim 5, wherein the robot control unit controls the rehabilitation robot so that if the physical state is perceived as an abnormal condition, a walking state of the rehabilitation robot is stopped and the rehabilitation robot is driven within a predefined stable condition.

10. The apparatus of claim 9, wherein the robot control unit provides a warning signal via the display unit if the physical state is beyond the predefined stable condition.

11. The apparatus of claim 1, further comprising a wearing-type suit having the apparatus for controlling a rehabilitation robot disposed thereon.
